(19) United States
(12) Patent Application Publication
(10) Pub. No.: US 2023/0407337 A1
(43) Pub. Date: Dec. 21, 2023

(54) CRISPR RIBONUCLEOPROTEIN COMPLEX GENOME EDITING OF HUMAN INNATE IMMUNE CELLS

(71) Applicant: The Regents if the University of California, Oakland, CA (US)

(72) Inventors: Timothy O'Sullivan, Los Angeles, CA (US); Luke Riggan, Los Angeles, CA (US); Andrew Hildreth, Los Angeles, CA (US)

(73) Assignee: The Regents if the University of California, Oakland, CA (US)

(21) Appl. No.: 18/254,086

(22) PCT Filed: Dec. 8, 2021
(86) PCT No.: PCT/US21/62365

§ 371 Date: May 23, 2023

Related U.S. Application Data
(60) Provisional application No. 63/122,553, filed on Dec. 8, 2020.

(51) Int. Cl. C12N 15/87; C12N 9/22; C12N 15/11; C12N 15/90; C12N 5/0786; C12N 5/0789; C12N 5/0783

(52) U.S. Cl. CPC C12N 15/87; C12N 9/22; C12N 15/111; C12N 15/907; C12N 5/0645; C12N 5/0647; C12N 5/0646; C12N 2310/20; C12N 2800/80

(57) ABSTRACT

The invention provides an optimized electroporation strategy for non-viral CRISPR-Cas9 ribonucleoprotein (cRNP) genomic editing of primary innate immune cells, a methodology that can, for example, produce an almost complete loss of target gene expression from a single electroporation. This methodology has been validated in human peripheral blood-derived monocyte derived macrophages, natural killer cells, and monocyte derived dendritic cells. This gene editing technology can, for example, be used to delete inhibitory molecules in natural killer cells and dendritic cells for adoptive cell therapy in cancer. It can also be used to manipulate gene expression in adoptively transferred tolorogenic dendritic cells for treatment of type 1 diabetes and other autoimmune diseases.

### CROSS-REFERENCE TO RELATED APPLICATIONS

This application claims the benefit under 35 U.S.C. Section 119(e) of co-pending and commonly-assigned. U.S. Provisional Patent Application Ser. No 63/122,553, filed on Dec. 8, 2020, and entitled “CRISPR RIBONUCLEOPROTEIN COMPLEX GENOME EDITING OF HUMAN INNATE IMMUNE CELLS” which application is incorporated by reference herein.

### STATEMENT REGARDING FEDERALLY SPONSORED RESEARCH AND DEVELOPMENT

This invention was made with government support under Grant Number AI145997, awarded by the National Institutes of Health. The government has certain rights in the invention.

### TECHNICAL FIELD

Embodiments of the disclosure concern at least the fields of immunology, cell biology, molecular biology, and medicine.

### BACKGROUND OF THE INVENTION

As is known in the art, a genome engineering tool has been developed based on the components of the type II prokaryotic CRISPR (Clustered Regularly Interspaced Short palindromic Repeats) adaptive immune system of the bacteria Streptococcus pyogenes. This multi-component system referred to as RNA-guided Cas nuclease system or more simply as CRISPR, involves a Cas endonuclease coupled with guide RNA molecules that have the ability to drive said nuclease to some specific genome sequences. Where the RNA guide hybridizes to the genome sequence, the endonuclease has the ability to cleave the DNA. The CRISPR/CRISPR-associated (Cas) system involves 1) retention of foreign genetic material, called “spacers”, in clustered arrays in the host genome, 2) expression of short guiding RNAs (crRNAs) from the spacers, 3) binding of the crRNAs to specific portions of the foreign DNA called protospacers and 4) degradation of protospacers by CRISPR-associated nucleases (Cas). The specificity of binding to the foreign DNA is controlled by the non-repetitive spacer elements in the pre-crRNA, which upon transcription along with the tracrRNA, directs the Cas9 nuclease to the protospacer:crRNA heteroduplex and induces double-strand breakage (DSP) formation.

CRISPR genome engineering has become a powerful tool to functionally investigate the complex mechanisms of various biological processes such as immune system regulation. While decades of work have aimed to genetically reprogram innate immunity, the utility of current approaches are restricted by poor knockout efficiencies or have limited specificity for primary leukocyte lineages in vivo.

In view of the above, methods that can use non-viral CRISPR-Cas9 ribonucleoprotein (cRNP) for the genomic editing of primary innate immune cells are needed.

### SUMMARY OF THE INVENTION

As discussed below, we have designed an optimized strategy for the non-viral CRISPR-Cas9 ribonucleoprotein (cRNP) genomic editing of primary innate immune cells. In illustrative working embodiments of the invention, fresh peripheral blood derived CD 14+monocytes or IL-15 stimulated CD56+ Natural Killer cells were electroporated under conditions comprising 1900 volts (V), with a pulse width of 1×20 milliseconds (ms). In such embodiments, the primary innate immune cells were electroporated in the presence of a single or multiple CRISPR ribonucleoprotein complexes to achieve gene deletion(s). In these working embodiments of the invention, we show that this methodology can produce an almost complete loss of target gene expression from a single electroporation.

The invention disclosed herein has a number of embodiments. Embodiments of the invention include, for example, methods of electroporating a CRISPR ribonucleoprotein complex into human primary leukocytes. Typically, these methods comprise combining the CRISPR ribonucleoprotein complex with the human primary leukocytes; and then electroporating this combination under conditions comprising: a voltage between 1700-2000 volts; and a pulse width of width of about 1×20-30 milliseconds; such that the CRISPR ribonucleoprotein complex is electroporated into the human primary leukocytes.

Typically in these methods, the CRISPR ribonucleoprotein complex comprises from 40 to 100 pmol Cas9 complexed with from 120 to 300 pmol sgRNA. In certain embodiments of the invention, these methods comprise not more than 1, 2 or 3 individual electroporations. Typically in these methods, the electroporation of the CRISPR ribonucleoprotein complex into the primary leukocytes results in modulation of expression of a gene in the leukocytes targeted by the sgRNA.

In illustrative embodiments of the invention, the primary leukocytes are collected from an individual, and are then cultured for one or more specific time periods, for example, between 1 and 21 days, following collection and prior to electroporation. Optionally in these methods, the primary leukocytes are combined with one or more cytokines in culture following collection and prior to electroporation. For example, in certain embodiments of the invention, the one or more cytokines is selected from: IL-2, IL 3, 1L-4, IL-15, the notch ligand DLL1, stem cell factor (SCF) , FLT3 ligand (FLT3L), thrombopoietin (TPO), GM CSF, and M-CSF. The primary leukocytes used in embodiments of the invention can be collected by any one of a number of art accepted practices. In some embodiments of the invention, the primary leukocytes are collected from an individual by a method comprising: administering to the individual a mobilization agent such as plerixafor, filgrastim, or a combination thereof so that leukocytes present in bone marrow in the individual are mobilized into the peripheral blood; and then collecting the leukocytes from peripheral blood of the individual.

In certain embodiments of the invention, the collected primary leukocytes are at least partially purified into one or more groups following their collection (e.g. monocytes, hematopoietic stem cells, Natural Killer cells and the like). For example, in certain embodiments of the invention, the one or more groups comprises: CD14+ monocytes; CD34+ hematopoietic stem cells; and/or CD56+ Natural Killer cells. As noted above, collected cells can then be cultured for one or more specific time periods of between 0 and 21 days following collection and prior to electroporation. Typically in these methods, the time that the primary leukocytes are cultured prior to electroporation is cell type specific. For example, in some embodiments of the invention, CD14+ monocytes are electroporated within 24 hours following their collection and prior to electroporation. In other embodiments of the invention, CD34+ hematopoietic stem cells are cultured for at least 3 days following their collection and prior to electroporation. In other embodiments of the invention, CD56+ Natural Killer cells are cultured for at least 3-17 days following their collection and prior to electroporation.

In certain embodiments of the invention, electroporation of the CRISPR ribonucleoprotein complex into the primary leukocytes inactivates a gene targeted by the sgRNA. In this way, embodiments of these methods allow for any gene deletion in primary human innate immune cells. To date this method has been validated in human peripheral blood-derived monocyte derived macrophages, natural killer cells, and monocyte derived dendritic cells. This gene editing technology can, for example, be used to delete inhibitory molecules in natural killer cells and dendritic cells for adoptive cell therapy in cancer. It can also be used to manipulate gene expression in adoptively transferred tolorogenic dendritic cells, for example, in the treatment of type 1 diabetes and other autoimmune diseases

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1C. Data from studies on CD11b⁺ macrophages. FIG. 1A shows a data flowchart of a gating strategy for the analysis of CD11b+ macrophages. FIG. 1B shows graphs of data on the expression of CD11b on macrophages after editing at D0, D1 and D2. This data indicates that CD11b editing on day 0 had a slightly higher editing efficiency. FIG. 1C shows graphs of CD11b editing efficiency (left panel) and viability (right panel) on macrophages after D0/1/2 editing.

FIG. 2. Data from studies on dendritic cells (DCs). FIG. 2 shows a data flowchart of a gating strategy for the analysis of dendritic cells.

FIG. 3. Data from studies on type one conventional DCC (cDC1). The left most panel provides graphed data showing the expression of CD45 on cDC1 cells at Day 0 electroporation, the two middle panels provide graphed data showing the expression of CD45 on cDC1 cells at Day 3 and day 7 electroporation, and the right panel shows graphed data showing the expression of CD45 on cDC1 cells at Days 0, 3 and 7. This data indicates that Electroporation didn't work on CD34-F with overnight pre stimulation and that both day 3 and day 7 worked and day 3 electroporation has higher efficiency.

FIG. 4, Data showing different editing efficiencies in different cell types. The left panel shows CD45⁺ expression in CD45 KO cells at day 0, the middle panel shows CD45⁺ expression in CD45 KO cells at day 4 and the right panel shows CD45⁺ expression in CD45 KO cells at day 7.

FIG. 5. Data from studies on CD14+ monocytes, FIG. 5 shows a data flowchart of a gating strategy for the analysis of CD14+ monocytes. In these studies, CD14+ Monocytes were isolated from donor PBMCs, and then cultured in 100 ng/ml. GM-CSF and 20 ng/mL IL-4 for 6 days.

FIGS. 6A-6G, Data from electroporation parameter studies on CD11b⁺ monocytes. FIG. 6A shows data from studies of voltage viability. In FIGS. 6A-6F, isolated CD14+ Monocytes were electroporated using 1×20 ms pulse using 40 pMol Cas9 at the voltages indicated: Cas9 only refers to electroporation without RNP. Cells were cultured in 100 ng/mL GM-CSF and 20 mg/mL IL-4 for 6 days. FIG. 6B shows data from studies of voltage editing. FIG. 6C shows data from studies of pulse code viability. FIG. 6D shows data from studies of pulse code editing efficiency. FIG. 6E shows data from studies of Cas9 viability FIG. 6F shows data from studies of Cas9 editing. FIG. 6G shows data from studies of cell viability (left panel) and CD11b+ expression (right panel) in cells exposed to different pulse codes.

FIG. 7, Data from electroporation parameter studies on CD11b⁺ monocytes. These data and the other disclosure provided herein provide evidence that electroporation at 1900V using 1×20 ms pulse and 40 pMol maximizes viability and editing efficiency. Using an alternate guide for CD11b, we can confirm these editing conditions: In these studies, isolated CD14+ Monocytes were electroporated 1900V using 1×20 ms pulse and 40 pMol. Cells were cultured in 100 ng/mL GM-CSF and 20 ng/mL IL-4 for 6 days.

FIG. 8. FACS Data studies of CD56 and CD 45 human NK cells. These studies show day 17 FACS data from Human NK (Donor 2) cultured in 20 ng/mL IL-15 and 1000 IU/ml, IL-2.

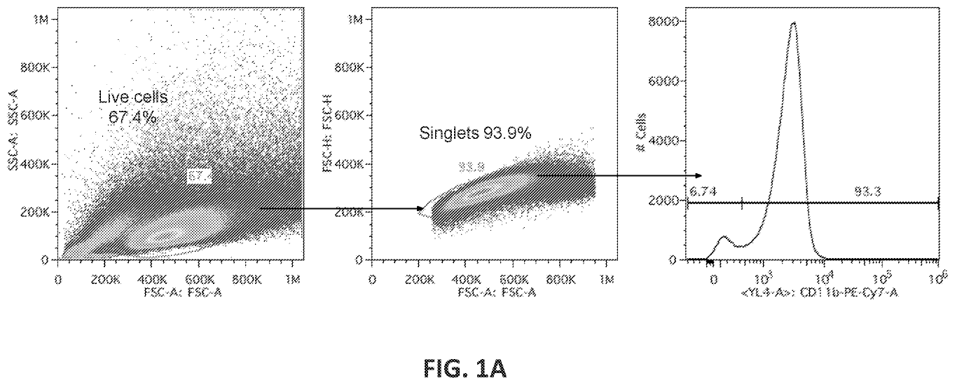

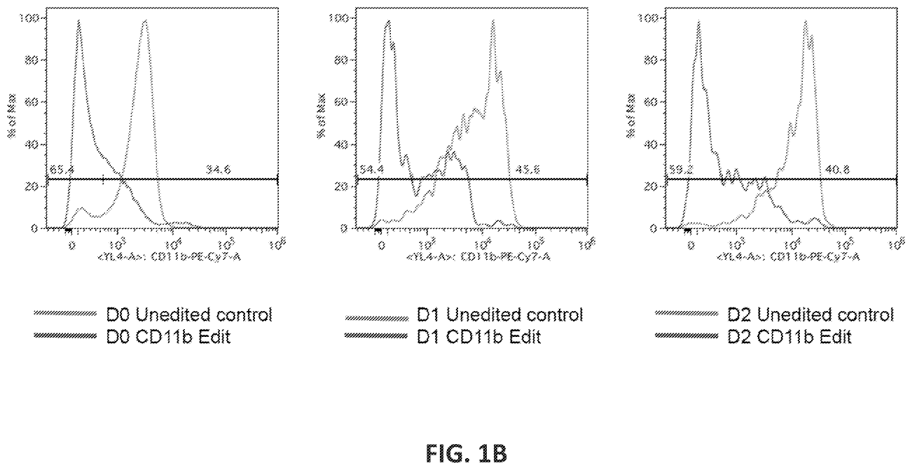

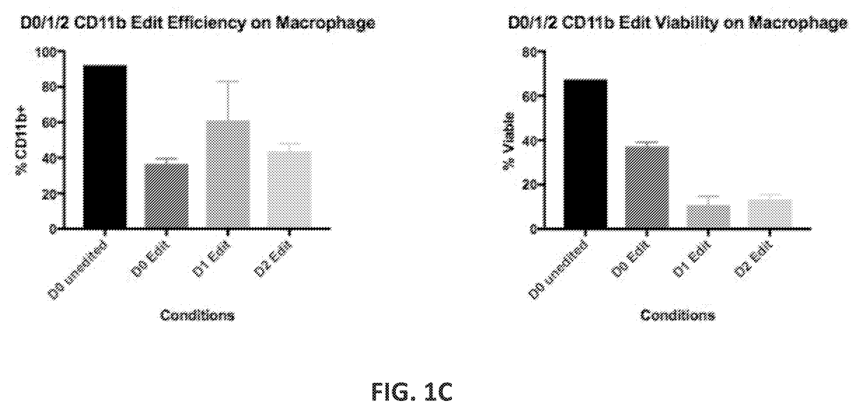

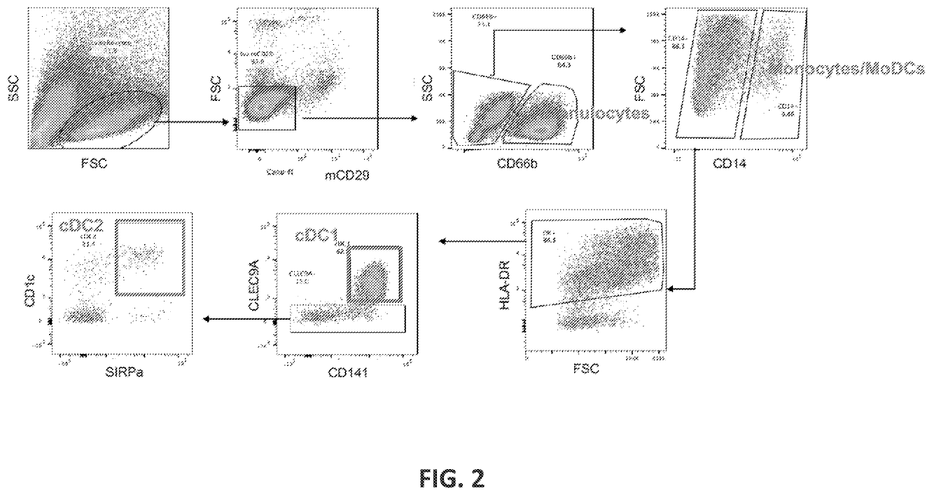

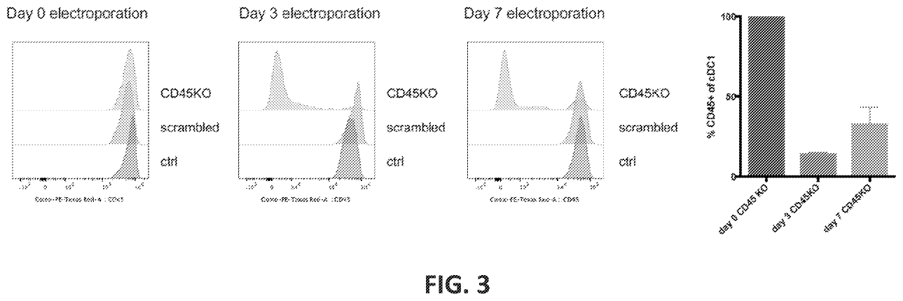

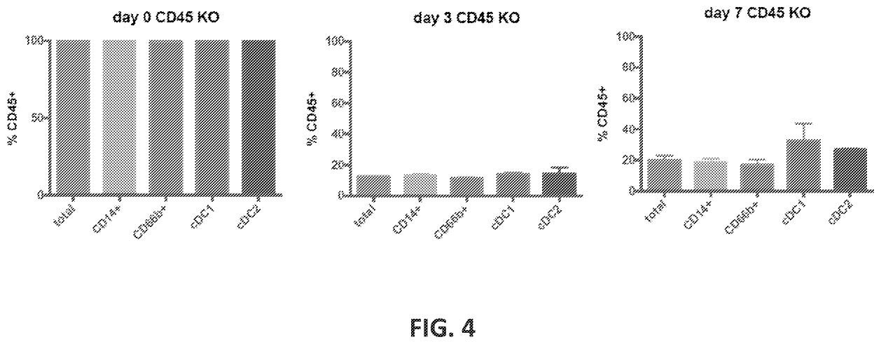

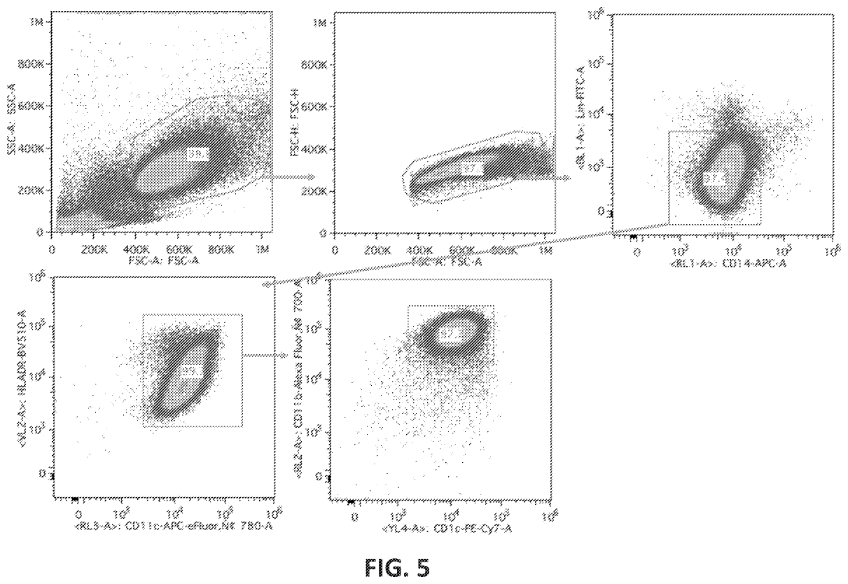

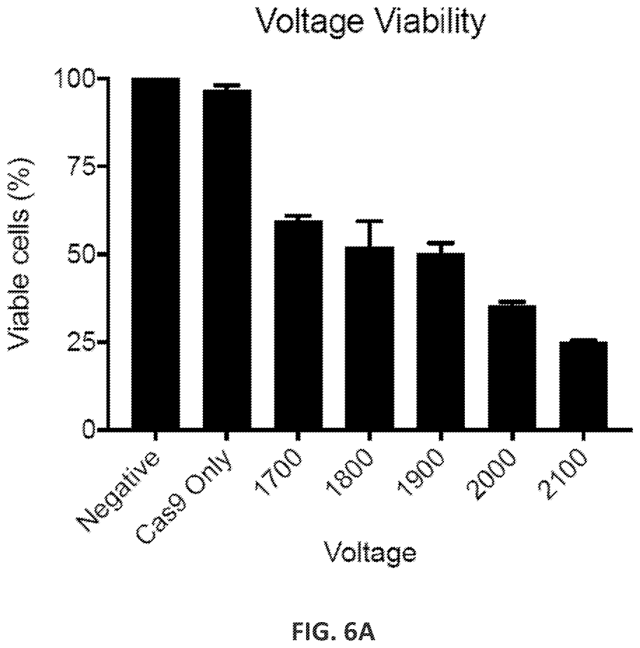

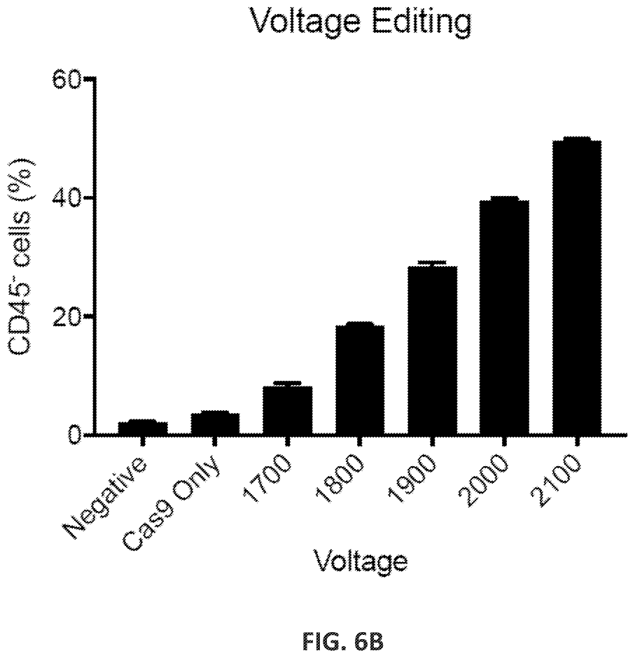

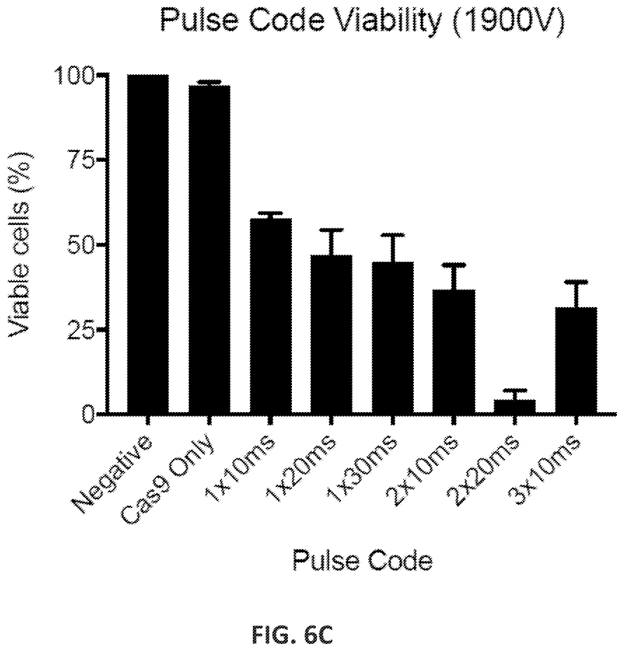

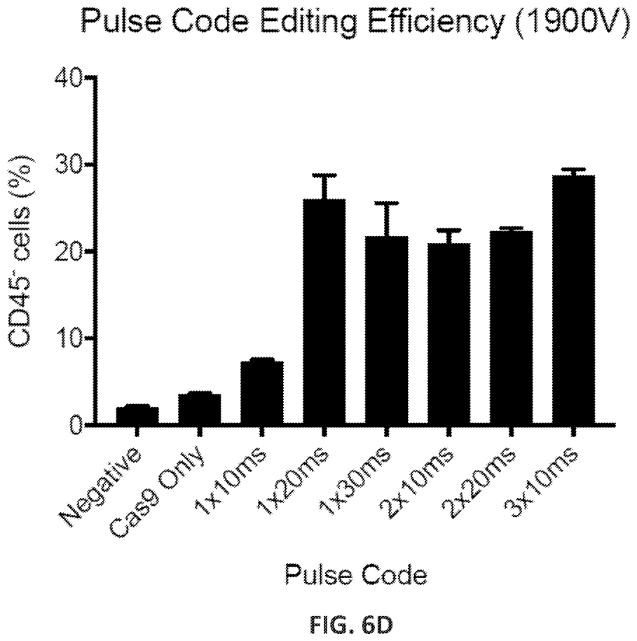

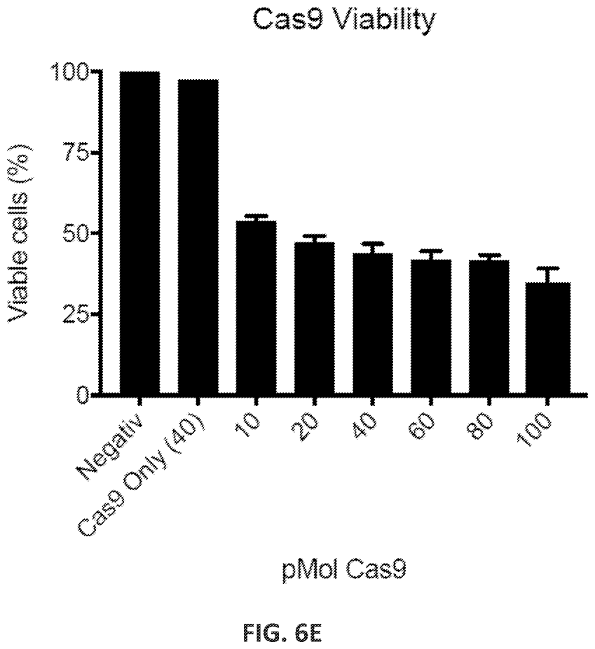

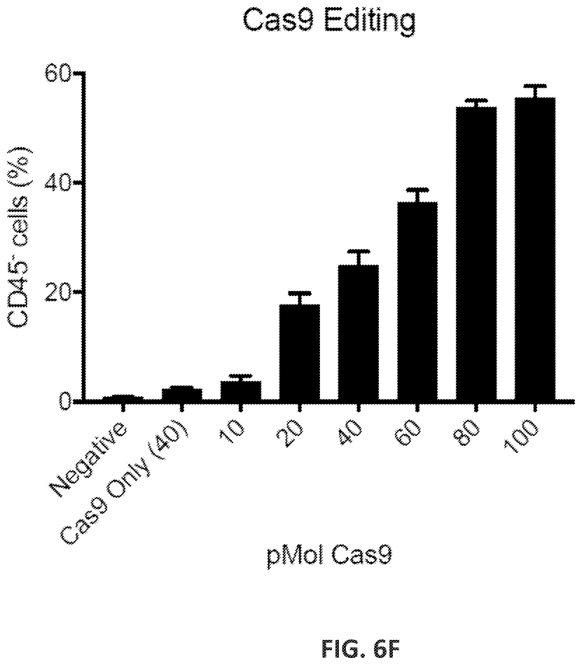

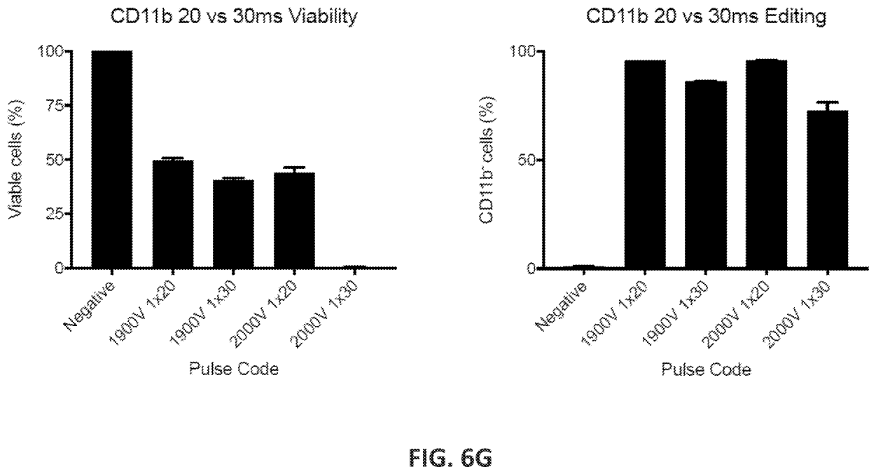

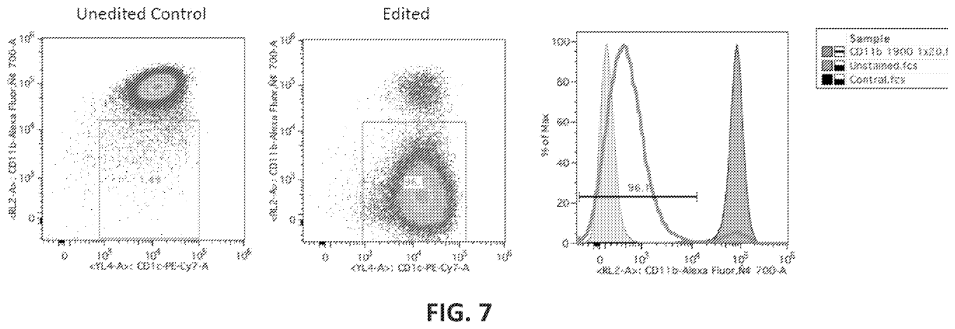

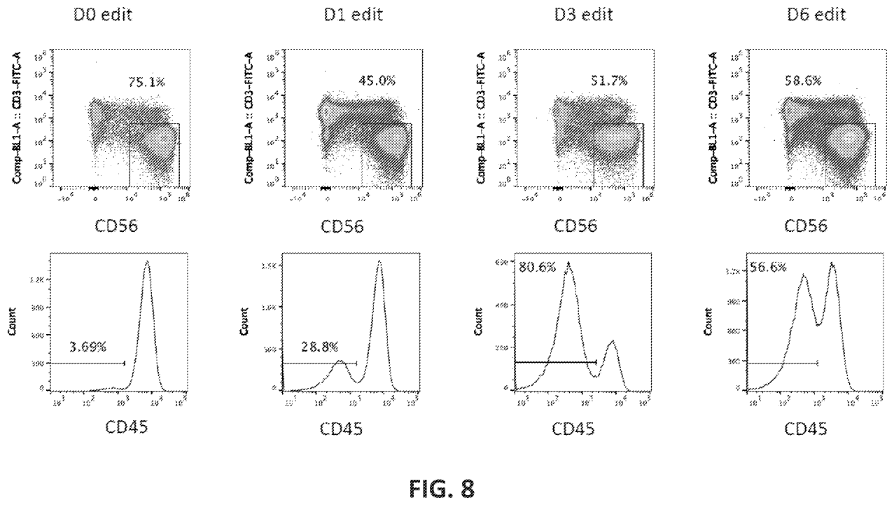

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

As discussed below, embodiments of the invention include, for example, methods of electroporating a CRISPR ribonucleoprotein complex into human primary leukocytes. As used herein, the phrase “CRISPR, ribonucleoprotein complex” refers to a ribonucleoprotein complex having CRISPR-associated endonuclease activity. Exemplary CRISPR ribonucleoprotein complexes include CRISPR/Cas9 CRISPR-associated endonuclease activity and CRISPR/Cpfl CRISPR-associated endonuclease activity. CRISPR/Cas9 gene targeting requires a custom single-lead RNA (sgRNA) consisting of a targeted sequence (crRNA sequence) and a Cas9 nucleic acid recruitment sequence (tracrRNA). The crRNA region is a sequence of about 20 nucleotides, homologous to one of the regions of the gene you are interested in, that will guide the activity of the Cas9 nuclease. As used herein, the phrase “CRISPR-associated RNA” refers to an RNA component that, when combined with a CRISPR-associated protein, results in an CRISPR ribonucleoprotein complex. Exemplary CRISPR ribonucleoprotein complexes include ribonucleoprotein complexes having an CRISPR-associated protein, such as CRISPR/Cas9 protein or CRISPR/Cpfl protein. An exemplary CRISPR-associated RNA includes a gRNA, including a crRNA and tracrRNA, for CRISPR/Cas9 protein that fours the CRISPR/Cas9 endonuclease system. Another exemplary CRISPR-associated RNA includes a crRNA for CRISPR/Cpfl protein that forms the CRISPR/Cpfl endonuclease system. Examples of these CRISPR ribonucleoprotein complexes, the CRISPR-associated RNA and protein components, and CRISPR-associated endonuclease systems are disclosed in the following references: Collingwood, M. A., Jacobi, A. M., Rettig, G. R, Schubert, M. S., and Behlke, M. A., “CRISPR-BASED COMPOSITIONS AND METHOD OF USE,” U.S. patent application Ser. No. 14/975,709, filed Dec. 18, 2015, published now as U.S. Patent Application Publication No. US2016/0177304A1 on Jun. 23, 2016 and issued as U.S. Pat. No. 9,840,702 on Dec. 12, 2017: and Behlke, M. A, et al. “CRISPR/CPF1 SYSTEMS AND METHODS,” U.S. patent application Ser. No, 15/821,736, filed Nov. 22, 2017. and U.S. Patent Application Publication No. 20190032131, the contents of which are hereby incorporated by reference herein in their entirety.

As is known in the art, primary cells (e.g. primary leukocytes) are those directly removed from an individual (e.g. a cancer patient), as compared to cell lines which are permanently established cell cultures. Typically, the methods of the invention comprise combining the CRISPR ribonucleoprotein complex with the primary leukocytes; and then electroporating this combination under conditions comprising: a voltage between 1700-2000 volts; and a pulse width of width of about 1×20-30 milliseconds; such that the CRISPR ribonucleoprotein complex is electroporated into the leukocytes. These parameters are used in these methods because our studies have determined that these electroporation parameters are unexpectedly effective for introducing a CRISPR ribonucleoprotein complex into primary leukocytes.

Typically in these methods, the CRISPR ribonucleoprotein complex comprises from 40 to 100 pmol Cas9 complexed with from 120 to 300 pawl sgRNA. In this context, a gRNA is comprised of a tracrRNA and crRNA. In particular, the crRNA and tracrRNA can be fused into a single chimeric nucleic acid (a single-guide RNA, or sgRNA) or they can be separate nucleic acids. In certain embodiments of the invention, these methods comprise not more than 1, 2 or 3 individual electroporations. Typically in these methods, the electroporation of the CRISPR ribonucleoprotein complex into the primary leukocytes results in modulation of expression of a gene in the leukocytes targeted by the sgRNA. Electroporation methods, materials and devices that can be used with embodiments of the invention are disclosed, for example in US Patent Application Publication Nos.: 20200332276, 20200171303, 20200131500, 20200115668, 20200048600, 20200048599, 20190382792, 20190292510, 20190284579, 20190125165, 20190100721, 20190093125, 20180340186, 20180179485, 20180155688, 20180066222, 20180064073, 20170348525, 20170298390, 20170218355, 20160215297, and 20160129246, the contents of which are incorporated herein by reference.

In illustrative embodiments of the invention, the primary leukocytes are collected from an individual, and are then cultured for one or more specific time periods of between 1 and 21 days (e.g. at least 1, 2, 3 . . . up to 21 days, or not more than 1, 2, 3 . . . up to 21 days, and/or from 1-2 or 2-3 or 3-5 or 3-7 or 5-10 or more days etc.) following collection and prior to electroporation. Optionally in these methods, the leukocytes are combined with one or more cytokines in culture following collection and prior to electroporation. For example, in certain embodiments of the invention, the one or more cytokines is selected from: IL-2, IL-3, IL-4, IL-15, the notch ligand DLL1 stem cell factor (SCF), FLT3 ligand (FLT3L), thrombopoietin (TPO), (IM-CSF, and M-CSF. In this context, illustrative methods and materials for such culture conditions are described, for example, in U.S. Patent Application Publication No. 2018/0155688, the contents of which are incorporated herein by reference. The primary leukocytes used in embodiments of the invention can be collected by any one of a number of art accepted practices. In some embodiments of the invention, the primary leukocytes are collected from an individual by a method comprising: administering to the individual a mobilization agent such as plerixafor, filgrastim, or a combination thereof so that leukocytes present in bone marrow in the individual are mobilized into the peripheral blood; and then collecting the leukocytes from peripheral blood of the individual (e.g. via apheresis).

In certain embodiments of the invention, the collected primary leukocytes are at least partially purified into one or more groups (e.g. monocytes, hematopoietic stem cells,

Natural Killer cells and the like) following their collection. For example, in certain embodiments of the invention, the one or more groups comprises: CD14+ monocytes; CD34+ hematopoietic stem cells; and/or CDS6+ Natural Killer cells. As noted above, collected cells can then be cultured for one or more specific time periods of less than one day or from 1 to 21 days following collection and prior to electroporation. Surprisingly, electroporation efficiency in different types of primary leukocytes can be optimized by culturing each of different types of primary leukocytes for different selected periods of time. For this reason, in some embodiments of the invention, CD14+ monocytes are electroporated within 24 or 45 hours following their collection and prior to electroporation. Similarly, in some embodiments of the invention, CD34+ hematopoietic stem cells are cultured for at least 3 days following their collection and prior to electroporation. Similarly, in some embodiments of the invention, CD56+ Natural Killer cells are cultured for at least 3-17 days following their collection and prior to electroporation.

One illustrative working embodiment of the invention is a method of electroporating a CRISPR ribonucleoprotein complex comprising from 40 to 100 pmol Cas9 complexed with from 12.0 to 300 pmol sgRNA into human CD14+ monocytes. This methodological embodiment of the invention comprises: collecting leucocytes from an individual; partially purifying the leucocytes to generate a population of cells enriched for CD14+ monocytes; combining the CRISPR ribonucleoprotein complex with the enriched population of cells and electroporating the CD14+ monocytes within 24 hours of collection from the individual; wherein electroporating the CRISPR ribonucleoprotein complex that has been combined with the enriched population of cells occurs under conditions comprising: a voltage between 1700-2000 volts; and a pulse width of width of about 1×20-30 milliseconds; such that the CRISPR ribonucleoprotein complex is electroporated into the CD14+ monocytes.

Another illustrative working embodiment of the invention is a method of electroporating a CRISPR ribonucleoprotein complex comprising from 40 to 100 pmol Cas9 complexed with from 120 to 300 pmol sgRNA into human CD34+ hematopoietic stem cells. This methodological embodiment of the invention comprises: collecting leucocytes from an individual; partially purifying the leucocytes to generate a population of cells enriched for CD34+ hematopoietic stem cells; culturing the CD34+ hematopoietic stem cells for at least 3 days prior to electroporation; combining the CRISPR ribonucleoprotein complex with the enriched population of cells; and then electroporating the CRISPR ribonucleoprotein complex that has been combined with the enriched population of cells under conditions comprising: a voltage between 1700-2000 volts; and a pulse width of width of about 1×20-30 milliseconds; such that the CRISPR ribonucleoprotein complex is electroporated into the CD34+ hematopoietic stem cells.

Another illustrative working embodiment of the invention is a method of electroporating a CRISPR ribonucleoprotein complex comprising from 40 to 100 pmol Cas9 complexed with from 120 to 300 pmol sgRNA into human CD56+ Natural Killer cells. This methodological embodiment of the invention comprises: collecting leucocytes from an individual; partially purifying the leucocytes to generate a population of cells enriched for CD56+ Natural Killer cells; culturing the CD56+ Natural Killer cells for at least 3-17 days prior to electroporation; combining the CRISPR ribonucleoprotein complex with the enriched population of cells; and then electroporating the CRISPR ribonucleoprotein complex that has been combined with the enriched population of cells under conditions comprising: a voltage between 1700-2000 volts; and a pulse width of width of about 1×20-30 milliseconds; such that the CRISPR ribonucleoprotein complex is electroporated into the CD56+ Natural Killer cells.

In certain embodiments of the invention, electroporation of the CRISPR ribonucleoprotein complex into the leukocytes inactivates a gene targeted by the sgRNA. in this way, embodiments of these methods allow for any gene deletion in primary human innate immune cells. To date this method has been validated in human peripheral blood-derived monocyte derived macrophages, natural killer cells, and monocyte derived dendritic cells. This gene editing technology can further be used to delete inhibitory molecules in natural killer cells and dendritic cells for adoptive cell therapy in cancer. It can also be used to manipulate gene expression in adoptively transferred tolorogenic dendritic cells, for example, in the treatment of type 1 diabetes and other autoimmune diseases.

In one illustrative working embodiment of the invention, human peripheral blood mononuclear cells (PBMCs) were obtained fresh from an individual and CD14+ monocytes were isolated. In a Day 0 edit step, these cells were used in the methodology so that: 1 Million cells were edited immediately after isolation with a CRISPR CD11 b sgRNA guide and electroporated at 1900V for 20 milliseconds. After resting for 1.5 hours, these cells were plated onto non-tissue culture treated 12-well plates at 500,000 cells/well in 1.5 mL high glucose DMEM with 10 ng/mL human M-CSF. In a Day ½ Edit step, CD14+ monocytes were plated with the same condition as above on Day 0. On D1/2, the cells were harvested with cell scrapers for editing (same editing condition as Day 0) and plated onto new plates. We then perform a media change 3 days after editing. All cells were harvested for flow cytometry analysis on Day 5.

In another illustrative working embodiment of the invention, G-CSF-mobilized peripheral blood (MPS) CD34+ HSPCs were thawed and pre-stimulated overnight (day 0) or for 3 days (day 3) with SCF/FLT3L/TPO/L-3 and plated in cDC1 conditions (96-well plate) on MS5-hDLL 1 stromal cells. Cells were electroporated at day 0, 3, and 7 (500k cells each timepoint). For D7 timepoint, cells were harvested and stromal cells depleted by MACS prior to electroporation. Cells were re-plated on new stromal cells at the same density at harvest. All conditions were harvested and analyzed at D21. Conditions are as follows:

Conditions:

| Day 0 | Day 3 | Day 7 |
|---|---|---|
| Control | Control | Control |
| Scrambled | Scrambled | Scrambled |
| CD45 KO | CD45 KO | CD45 KO |
| Initial | Replated after 3 days | Replated post- |
| plating | of pre-stimulation | electroporation |

Cell # plated per well (−1:1 replating on D 3 and D 7)
Day 0: 5000 cells/well
Day 3: 30,000 cells/well
Day 7: 50,000 cells/well
All wells were analyzed on Day 21. For analysis, 3 replicate wells of each condition were collected and pooled.

Additional electroporation methods, materials and devices that can be used with embodiments of the invention are disclosed, for example in US Patent Application Publication Nos.: 20200362355, 20200048606, 20200000851 and 20190388469, as well as literature references such as: Functional CRISPR dissection of gene networks controlling human regulatory T cell identity. Schumann et al. Nat Immunol (2020); CRISPR screen in regulatory T cells reveals modulators of Foxp3; Cortez et al., Nature 2020, 29 Apr.; Pooled Knockin Targeting for Genome Engineering of Cellular Immunotherapies. Roth et al., Cell 2020 Apr. 30; Polymer-stabilized Cas9 nanoparticles and modified repair templates increase genome editing efficiency. Nguyen et al., Nat Biotechnol. 2019 Dec. 9; Landscape of stimulation-responsive chromatin across diverse human immune cells; Calderon et al., Nat Genet. 2019 Sep 30; Large dataset enables prediction of repair after CRISPR-Cas9 editing in primary T cells; Leenay et al., Nat Biotechnol. 2019 September;37(9):1.034-1037; A large CRISPR-induced bystander mutation causes immune dysregulation; Simeonov et al., Commun Biol. 2019 Feb 18;2:70; Genome-wide CRISPR Screens in Primary Human T Cells Reveal Key Regulators of Immune Function. Shifrut et al., Cell 2018 December; 175:1-14; Reprogramming human T cell function and specificity with non-viral genome targeting; Roth et al., Nature. 2018 July;559(7714):405-409; “T-bet”-ing on autoimmunity variants. Nguyen et al., PLOS Genetics. 13(9); e1006924 (2017); Discovery of stimulation-responsive immune enhancers with CRISPR activation; Simeonov et al., Nature. 549; 111-115 (2017); A Cas9 Ribonucleoprotein Platform for Functional Genetic Studies of HIV-Host Interactions in Primary Human Cells. Hulquist et al., Cell Reports,17; 138-1452 (2016); and Generation of Knock-in Primary Human T Cells Using Cas9 Ribonucleoproteins Schumamn. et al,, PNAS. (2015), the contents of all of which are incorporated herein by reference.

All publications mentioned herein (e.g. Riggan et al,, Cell Rep. 2020 May 19;31(7):107651; and the other references identified above) are incorporated by reference to disclose and describe aspects, methods and/or materials in connection with the cited publications.

Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of electroporating a CRISPR ribonucleoprotein complex into human primary leukocytes, the method comprising:
(a) combining the CRISPR ribonucleoprotein complex with the human primary leukocytes;
(b) electroporating the combination of (a) under conditions comprising:
a voltage between about 1700-2000 volts; and
a pulse width of width of at least about 1×20-30 milliseconds;
such that the CRISPR ribonucleoprotein complex is electroporated into the human primary leukocytes.

2. The method of claim 2, wherein the CRISPR ribonucleoprotein complex comprises from 40 to 100 pmol Cas9 complexed with from 120 to 300 pmol sgRNA.

3. The method of claim 2, wherein:
the leukocytes are collected from an individual;
the leukocytes are cultured for a time period of from 1 hour to up to 21 days following collection and prior to electroporation; and/or
the leukocytes are at least partially purified into one or more groups.

4. The method of claim 3, wherein the one or more groups comprises:
CD14+ monocytes;
CD34+ hematopoietic stem cells; and/or
CD56+ Natural Killer cells.

5. The method of claim 3, wherein the leukocytes are combined with one or more cytokines following collection and prior to electroporation; wherein the one or more cytokines is selected from: IL-2, IL-3, IL-4, IL-15, the notch ligand DLL1, stem cell factor (SCF) , FLT3 ligand (FLT3L), thrombopoietin (TPO), GM-CSF, and M-CSF.

6. The method of claim 3, wherein:
CD14+ monocytes are electroporated within 24 hours following collection;
CD34+ hematopoietic stem cells are cultured for at least 3 days following collection and prior to electroporation; and/or
CD56+ Natural Killer cells are cultured for at least 3-17 days following collection and prior to electroporation.

7. The method of claim 2, wherein electroporation of the CRISPR ribonucleoprotein complex into the leukocytes results in modulation of expression of a gene in the leukocytes targeted by the sgRNA.

8. The method of claim 7, wherein electroporation of the CRISPR ribonucleoprotein complex into the leukocytes inactivates a gene targeted by the sgRNA.

9. The method of claim 3, wherein the leukocytes are collected from an individual by a method comprising: administering to the individual a mobilization agent such as plerixafor, filgrastim, or a combination thereof so that leukocytes present in bone marrow in the individual are mobilized into the peripheral blood; and collecting the leukocytes from peripheral blood of the individual.

10. The method of claim 1, wherein the method comprises 1, 2 or 3 individual electroporations.

11. A method of electroporating a CRISPR ribonucleoprotein complex comprising from 40 to 100 pmol Cas9 complexed with from 120 to 300 pmol sgRNA into human CD14+ monocytes, the method comprising:
collecting leucocytes from an individual;
partially purifying the leucocytes to generate a population of cells enriched for CD14+ monocytes;
electroporating the CD14+ monocytes within 24 hours of collection from the individual;
combining the CRISPR ribonucleoprotein complex with the enriched population of cells;
electroporating the CRISPR ribonucleoprotein complex that has been combined with the enriched population of cells under conditions comprising:
a voltage between 1700-2000 volts; and
a pulse width of width of about 1×20-30 milliseconds;
such that the CRISPR ribonucleoprotein complex is electroporated into the CD14+ monocytes.

12. A method of electroporating a CRISPR ribonucleoprotein complex comprising from 40 to 100 pmol Cas9 complexed with from 120 to 300 pmol sgRNA into human CD34+ hematopoietic stem cells, the method comprising:
collecting leucocytes from an individual;
partially purifying the leucocytes to generate a population of cells enriched for CD34+ hematopoietic stem cells;
culturing the CD34+ hematopoietic stem cells for at least 3 days prior to electroporation;
combining the CRISPR ribonucleoprotein complex with the enriched population of cells;
electroporating the CRISPR ribonucleoprotein complex that has been combined with the enriched population of cells under conditions comprising:
a voltage between 1700-2000 volts; and
a pulse width of width of about 1×20-30 milliseconds;
such that the CRISPR ribonucleoprotein complex is electroporated into the CD34+ hematopoietic stem cells.

13. A method of electroporating a CRISPR ribonucleoprotein complex comprising from 40 to 100 pmol Cas9 complexed with from 120 to 300 pmol sgRNA into human CD56+ Natural Killer cells, the method comprising:
collecting leucocytes from an individual;
partially purifying the leucocytes to generate a population of cells enriched for CD56+ Natural Killer cells;
culturing the CD56+ Natural Killer cells for at least 3-17 days prior to electroporation;
combining the CRISPR ribonucleoprotein complex with the enriched population of cells;
electroporating the CRISPR ribonucleoprotein complex that has been combined with the enriched population of cells under conditions comprising:
a voltage between 1700-2000 volts; and
a pulse width of width of about 1×20-30 milliseconds;
such that the CRISPR ribonucleoprotein complex is electroporated into the CD56+ Natural Killer cells.

14. The method of claim 11, wherein the wherein electroporation of the CRISPR ribonucleoprotein complex into the CD14+ monocytes, the CD34+ hematopoietic stem cells, or the CD56+ Natural Killer cells inactivates a gene targeted by the sgRNA.

15. The method of claim 11, wherein the CD14+ monocytes, the CD34+ hematopoietic stem cells, or the CD56+ Natural Killer cells are combined with one or more cytokines following collection and prior to electroporation; wherein the one or more cytokines is selected from: IL-2, IL-3, IL-4, IL-15, the notch ligand DLL1, stem cell factor (SCF) , FLT3 ligand (FLT3L), thrombopoietin (TPO), GM-CSF, and M-CSF.
